(19) United States
(12) Patent Application Publication
(10) Pub. No.: US 2010/0179192 A1
(43) Pub. Date: Jul. 15, 2010

(54) USE OF NITRIC OXIDE RELEASING COMPOUNDS IN THE TREATMENT OF CHRONIC PAIN

(75) Inventors: Francesco Impagnatiello, Milano (IT); Daniela Ronchetti, Como (IT); Ennio Ongini, Segrate (IT); Francesca Benedini, San Donato Milanese (IT)

Correspondence Address: ARENT FOX LLP, 1050 CONNECTICUT AVENUE, N.W., SUITE 400, WASHINGTON, DC, 20036, US

(73) Assignee: NICOX S.A., Sophia Antipolis - Valbonne (FR)

(21) Appl. No.: 12/663,497

(22) PCT Filed: Jun. 24, 2008
(86) PCT No.: PCT/EP2008/057987

§ 371 Date: Dec. 7, 2009

(30) Foreign Application Priority Data
Jul. 9, 2007 (EP) 07013376.4

(51) Int. Cl. A61K 31/21; C07C 203/00; A61K 31/4535; A61P 29/00

(52) U.S. Cl. 514/326; 558/482; 514/509

(57) ABSTRACT

The present invention relates to nitrooxyderivative of antioxidant compounds of formula (I) and pharmaceutically acceptable salts or stereoisomers thereof for the treatment of chronic pain, in particular chronic neuropathic pain. The invention also describes composition comprising a nitrooxyderivative of a antioxidant compound of formula (I) and an analgesic drugs.

### FIELD OF THE INVENTION

The present invention relates to the use of nitric oxide releasing antioxidant compounds for the treatment of chronic pain, in particular chronic neuropathic pain. The present invention also relates to compounds having an improved effectiveness in alleviation or/and in the treatment of chronic neuropathic pain.

### BACKGROUND OF THE INVENTION

It is known that neuropathic pain is a form of chronic pain arising from a damage or disease of the central or peripheral nervous system. Neuropathic pain comprises a series of painful symptomatologies such as diabetic neuropathic pain, painful post-infarct syndrome, pain caused by chemotherapeutic treatment or pain arising from infections by viral agents, for example herpes, Herpes zoster, etc.

Neuropathic pain generally affects patients for many years, and is a social problem in that symptoms chronicity induces in subjects serious psychological stress.

In the last twenty years, research on neuropathic pain pathogenesis has achieved significant advances. Studies carried out on human and animal models of neuropathic pain have shown that central nervous system reacts to algogen stimuli with a series of biochemical and physiopathologic responses. This ability of the central nervous system to functionally and morphologically adapt to algogen stimuli is known as neuroplasticity and plays an essential role in inducing onset or in maintaining the painful symptomatology.

Carbamazepine, that has been widely used in clinical studies, has shown to be active in treating trigeminal neuralgia, diabetic neuropathic pain, and post-herpetic neuralgia. The administration of this drug has the drawback to present side effects such as somnolence, dizziness, ataxy, nausea and vomiting, thus limiting its use (Martindale XXXth Ed, page 342).

In the last years, other drugs for the treatment of neuropathic pain have been used. Among these gabapentin, pregabalin and tiagabine can be mentioned as being active as analgesic drug for treating neuropathic pain, mainly against diabetic neuropathic pain and post-herpetic pain. However, also in this case, high dosages are needed, short duration of action and sometime serious adverse effects are important documented drawbacks associated with these compounds. For example somnolence, weariness, obesity, etc. have been observed following gabapentin treatment (Martindale XXXth Ed, page 374).

Despite these disadvantages, no new classes of analgesics have been developed recently; there is clearly a need for additional therapies for the alleviation or/and treatment of chronic neuropathic pain.

It has now been surprisingly found that nitric oxide releasing derivatives of antioxidant compounds have analgesic activities and they are effective as analgesic drugs in the treatment of chronic neuropathic pain.

It has been further surprisingly found that the nitrooxyderivatives of the present invention show an enhanced therapeutic effect in the treatment of neuropathic pain when used in combination with analgesic therapeutic agents compared to the use of either agent alone.

The activity of natural phenolic acids such as ferulic, caffeic, vanillic and cumaric acids as antioxidant drugs is known (Handbook of Antioxidants-Second Edition, 2002).

Another class of known antioxidant drugs is represented by hydroquinones (Martindale XXXth Ed, page 1115).

WO 02/092072 discloses that the nitrooxyderivates of ferulic acid are able to prevent the deposition of the amyloid plaques and to reduce the neurodegenerative process and therefore they can be used for the prevention or treatment of Alzheimer disease.

WO 2005/065361 discloses that nitrooxyderivatives of caffeic acid, resveratrol and phtahalic acid have antineoplastic properties.

WO 01/12584 discloses the antioxidant properties of the 4-nitroxybutyl ester of ferulic acid; the document also discloses the use of the nitrooxyderivative for the treatment of oxidative stress and endothelial dysfunctions.

However, neither of these patents describes the use of these compounds as specific analgesics for the treatment of chronic pain or chronic neuropathic pain.

WO 03/000642 discloses that the combination of nitric oxide releasing compounds with drugs for the treatment of chronic pain shows a synergic effect and therefore the use of these combinations for the treatment of chronic pain allows to reduce the amount of analgesic compound and consequently the side effects are reduced. WO 03/000642 does not disclose the analgesic properties of the nitrooxyderivatives of antioxidant compounds.

An object of the present invention is the use for the treatment of the chronic pain of compounds of formula (I)

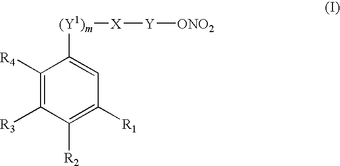

or pharmaceutically acceptable salts or stereoisomers thereof, wherein in formula (I)
m is an integer equal to 0 or 1;
Y¹ is —CH═CH— (CH₂)ₘ_{¹}—, wherein m¹ is an integer from 0 to 3, or —(CH₂)ₘ_{²}—, wherein m² is an integer from 1 to 3; preferably m¹ is 0, preferably m² is 2;
R₁, R₂, R₃, R₄ are independently selected from H, OH, —OR₅ wherein R₅ is a straight or branched (C₁-C₁₀)-alkyl, straight or branched C₁-C₂₀ alkyl, preferably —OR₅ is —OCH₃, with the proviso that at least one of R₁, R₂, R₃, R₄ is not H;
X is —OC(O)—, —OC(O)O—, —C(O)O—, —C(O)NR₆—, —C(O)S— wherein R₆ is H or a (C₁-C₅)-alkyl, preferably R₆ is H or —CH₃;
Y is a bivalent radical having the following meaning:
a) straight or branched C₁-C₂₀ alkylene optionally substituted with one or more substituents independently selected from halogen atoms, hydroxy, —ONO₂ or T, wherein T is —OC(O)—(C₁-C₁₀ alkyl)-ONO₂ or —O(C₁-C₁₀ alkyl)-ONO₂; preferably Y is a (C₁-C₁₀) alkylene or a (C₁-C₁₀) alkylene substituted with a —ONO₂;
   cycloalkylene with 5 to 7 carbon atoms into cycloalkylene ring, the ring being optionally substituted with side chains T₁, wherein T₁ is straight or branched C₁-C₁₀ alkyl, T₁ is preferably CH₃;

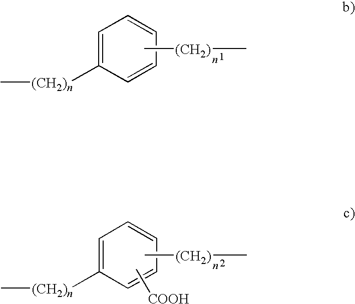

wherein n is an integer from 0 to 20, and n¹ is an integer from 1 to 20, preferably n is 0 or 1, preferably n¹ is an integer from 1 to 5;

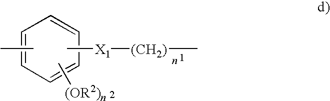

wherein
X₁ is —OC(O)— or —C(O)O—, n² is an integer from 1 to 3 and R² is H or CH₃;
n¹ is as defined above and n² is an integer from 0 to 2;

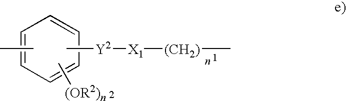

wherein:
Y² is —CH₂—CH₂— (CH₂)ₙ_{²}—; or —CH═CH— (CH₂)ₙ_{²}—;
X₁ is —OC(O)— or —C(O)O—, n² is an integer from 1 to 3 and R² is H or CH₃;
when Y is one of the bivalent radicals selected from b) to e), the —ONO₂ group is bound to —(CH₂)ₙ_{¹}— group;
when Y is one of the bivalent radicals selected from b) to e), m is 1;

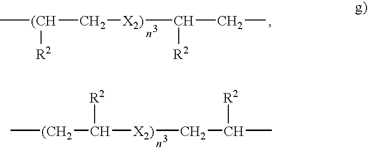

wherein X₂ is —O— or —S— or NR₆— wherein R₆ is as above defined, preferably X₂ is —O—;
n³ is an integer from 1 to 6, preferably from 1 to 4, more preferably n³ is 1, R² is as defined above, preferably R² is H;

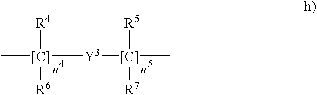

wherein:
n⁴ is an integer from 0 to 10;
n⁵ is an integer from 1 to 10;
R⁴, R⁵, R⁶, R⁷ are the same or different, and are H or straight or branched C₁-C₄ alkyl, preferably R⁴, R⁵, R⁶, R⁷ are H;
wherein the —ONO₂ group is linked to

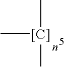

wherein n⁵ is as defined above;
Y³ is an heterocyclic saturated, unsaturated or aromatic 5 or 6 members ring, containing one or more heteroatoms selected from nitrogen, oxygen, sulfur, and is selected from

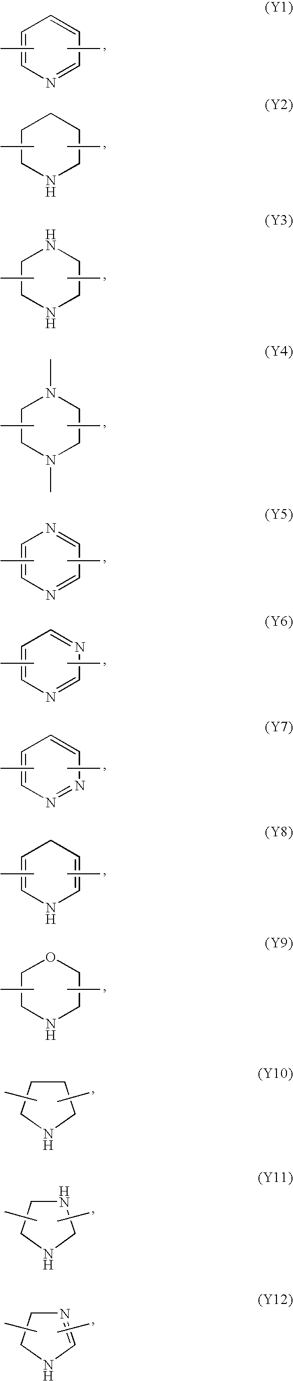

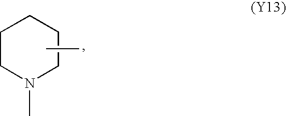

preferably Y³ is (Y4) or (Y13).

Preferably in formula (I):

R₁ is —OCH₃, R₂ is OH, R₃ and R₄ are H, m is 0 or m is 1 and Y¹ is —CH═CH—(CH₂)ₘ_{¹}, wherein m¹ is 0, or
R₁ and R₂ are OH, R₃ and R₄ are H, m is 0 or m is 1 and Y¹ is —CH═CH—(CH₂)ₘ_{¹}, wherein m¹ is 0, or Y¹ is —(CH₂)ₘ_{²}— wherein m² is 2; or
R₁, R₃ and R₄ are H and R₂ is OH, m is 0 or m is 1 and Y¹ is —CH═CH—(CH₂)ₘ_{¹}, wherein m¹ is 0 or, or Y¹ is —(CH₂)ₘ_{²}— wherein m² is 2;

### R₁, R₂ and R₃ are OH, R₄ is H and m is 0

### R₁ and R₃ are —OCH₃, R₂OH and R₄ is H, m is 0;

### R₁ is OH, R₂ is —OCH₃, R₃ and R₄ are H and m is 0;

### R₁ is OH, R₂ and R₃ are H, R₄ is —OCH₃, m is 0;

### R₁ and R₄ are OH, R₂ and R₃ are H, m is 0

### R₁ and R₃ are H and R₂ and R₄ are OH, m is 0.

The term “C₁-C₂₀ alkylene” as used herein refers to branched or straight C₁-C₂₀ hydrocarbon chain, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight chain alkyl groups comprising 1 to 10 carbon atoms, including methyl, ethyl, n-propyl, isopropyl, n-butyl, isobutyl, t-butyl, pentyl, hexyl, octyl and the like.

The term “cycloalkylene” as used herein refers to ring having from 5 to 7 carbon atoms including, but not limited to, cyclopentylene, cyclohexylene optionally substituted with side chains such as straight or branched (C₁-C₁₀)-alkyl, preferably CH₃.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile or tetrahydrofuran with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

While the natural antioxidant drugs have no effect on the treatment of neuropathic pain, the correspondent nitrooxyderivatives of formula (I) have shown to be active.

Another object of the present invention relates to combinations comprising a compound of formula (I) and at least one therapeutic agent used to treat neuropathic pain selected between the group of gabapentin, tiagabine and pregabalin and their use for treating chronic pain, in particular chronic neuropathic pain.

The combinations of the present invention are related to equimolar mixtures of the nitrooxyderivative of antioxidant drug of formula (I) and one analgesic drug selected from the group gabapentin, pregabalin and tiagabine. These combinations have proved to have a synergistic effect that has significant advantages in pharmacological activity. In particular, it has been recognized that the combinations have an improved pharmacological profile, with prolonged analgesic activity. Thus, the therapeutic efficacy in the treatment of the neuropathic pain of this combination of agents is enhanced relatively to the use of either agent alone. More particularly, the therapeutic efficacy is synergistically enhanced.

Another object of the invention relates to kits comprising at least a compound of formula (I) and at least one analgesic drug selected from the group gabapentin, pregabalin and tiagabine.

The daily dose of active ingredient that should be administered can be a single dose or it can be an effective amount divided into several smaller doses that are to be administered throughout the day. Usually, total daily dose may be in amounts preferably from 10 to 5000 mg of each compound. Preferred combinations include 150 to 1500 mg of nitrooxyderivatives of formula (I) and 100 to 1000 mg of an analgesic drug selected between the group of gabapentin, tiagabine and pregabalin. The dosage regimen and administration frequency for treating the mentioned diseases with the compound of the invention and/or with the pharmaceutical compositions of the present invention will be selected in accordance with a variety of factors, including for example age, body weight, sex and medical condition of the patient as well as severity of the disease, route of administration, pharmacological considerations and eventual concomitant therapy with other drugs. In some instances, dosage levels below or above the aforesaid range and/or more frequent may be adequate, and this logically will be within the judgment of the physician and will depend on the disease state.

Another object of the present invention relates to compounds of formula (I)

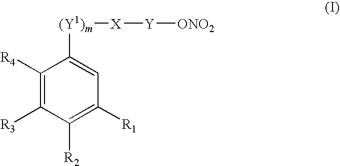

or pharmaceutically acceptable salts or stereoisomers thereof, wherein in formula (I)
m is an integer equal to 0 or 1;
Y¹ is —CH═CH—(CH₂)ₘ_{¹}, wherein m¹ is an integer from 0 to 3, or —(CH₂)ₘ_{²}— wherein m₂ is an integer from 1 to 3; preferably m¹ is 0, preferably m² is 2;
R₁, R₂, R₃, R₄ are independently selected from H, OH, —OR₅ wherein R₅ is a straight or branched (C₁-C₁₀)-alkyl, straight or branched C₁-C₂₀ alkyl, preferably —OR₅ is —OCH₃, with the proviso that at least one of R₁, R₂, R₃, R₄ is not H;
X is —OC(O)—, —OC(O)O—, —C(O)O—, —C(O)NR₆—, —C(O)S— wherein R₆ is H or a (C₁-C₅)-alkyl, preferably R₆ is H or —CH₃;
Y is a bivalent radical having the following meaning:
b) straight or branched C₁-C₂₀ alkylene optionally substituted with one or more substituents independently selected from halogen atoms, hydroxy, —ONO₂ or T, wherein T is —OC(O) (C₁-C₁₀ alkyl)-ONO₂ or —O(C₁-C₁₀ alkyl)-ONO₂; preferably Y is a (C₁-C₁₀) alkylene or a (C₁-C₁₀) alkylene substituted with a —ONO₂;
   cycloalkylene with 5 to 7 carbon atoms into cycloalkylene ring, the ring being optionally substituted with side chains T₁, wherein T₁ is straight or branched C₁-C₁₀ alkyl, T₁ is preferably CH₃;

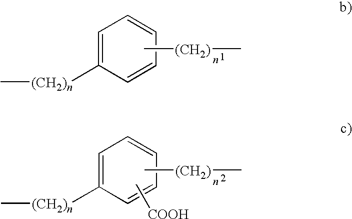

wherein n is an integer from 0 to 20, preferably n is an integer from 0 to 5,
n¹ is an integer from 1 to 20, preferably n¹ is an integer from 1 to 5;

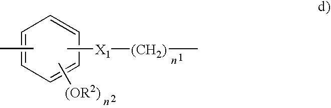

wherein
X₁ is —OC(O)— or —C(O)O—, n² is an integer from 1 to 3 and R² is H or CH₃;
n¹ is as defined above and n² is an integer from 0 to 2;

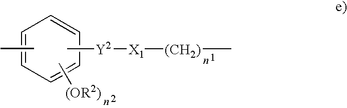

wherein:
Y² is —CH₂—CH₂—(CH₂)ₙ_{²}—; or —CH═CH—(CH₂)ₙ_{²}—;
X₁ is —OC(O)— or —C(O)O—, n² is an integer from 1 to 3 and R² is H or CH₃;
when Y is one of the bivalent radicals b) to e), the —ONO₂ group is bound to the —(CH₂)ₙ_{¹}— group;
when Y is one of the bivalent radicals mentioned under b) to
e), m is 1;

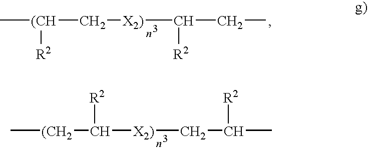

wherein X₂ is —O— or —S— or NR₆— wherein R₆ is as above defined, preferably X₂ is —O—, n³ is an integer from 1 to 6, preferably from 1 to 4, R² is as defined above, preferably R² is H;

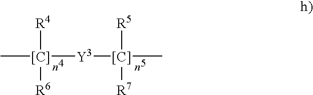

wherein:
n⁴ is an integer from 0 to 10, preferably n⁴ is 1;
n⁵ is an integer from 1 to 10, preferably n⁵ is an integer from 1 to 5;
R⁴, R⁵, R⁶, R⁷ are the same or different, and are H or straight or branched C₁-C₄ alkyl, preferably R⁴, R⁵, R⁶, R⁷ are H;
wherein the —ONO₂ group is linked to

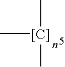

wherein n⁵ is as defined above;
Y³ is an heterocyclic saturated, unsaturated or aromatic 5 or 6 members ring, containing one or more heteroatoms selected from nitrogen, oxygen, sulfur, and is selected from

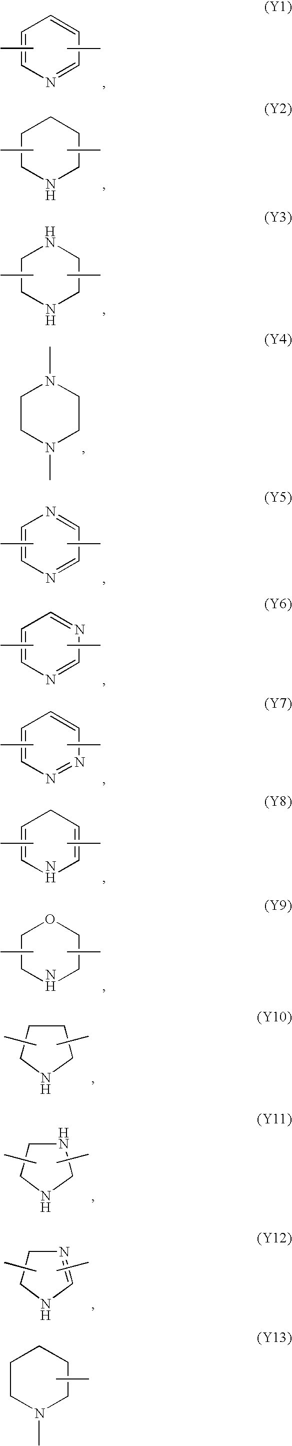

excluding:
(E)-3-(3,4-Dihydroxy-phenyl)-2-propenoic acid 3-(nitrooxy-methyl)phenyl ester;
(E)-3-(3,4-Dihydroxy-phenyl)-2-propenoic acid 2-(nitrooxy-methyl)phenyl ester;
(E)-3-(3,4-Dihydroxy-phenyl)-2-propenoic acid 4-(nitrooxy-methyl)phenyl ester;
(E)-3-(3,4-Dihydroxy-phenyl)-2-propenoic acid 3-(nitrooxy-methyl)phenyl amide;
(E)-3-(3,4-Dihydroxy-phenyl)-2-propenoic acid 2-(nitrooxy-methyl)phenyl amide;
(E)-3-(3,4-Dihydroxy-phenyl)-2-propenoic acid 4-(nitrooxy-methyl)phenyl amide;
(E)-3-(3,5-Dihydroxy-phenyl)-2-propenoic acid 3-(nitrooxy-methyl)phenyl ester;
(E)-3-(3,5-Dihydroxy-phenyl)-2-propenoic acid 3-(nitrooxy-methyl)phenyl amide;
(E)-3-(3-hydroxy-4-methoxy-phenyl)-2-propenoic acid 3-(nitrooxy-methyl)phenyl ester;
(E)-3-(3-hydroxy-4-methoxy-phenyl)-2-propenoic acid 3-(nitrooxy-methyl)phenyl amide;
(E)-3-(3,4-Dimethoxy-phenyl)-2-propenoic acid 3-(nitrooxy-methyl)phenyl ester;
(E)-3-(3,4-Dimethoxy-phenyl)-2-propenoic acid 2-(nitrooxy-methyl)phenyl ester;
(E)-3-(3,4-Dimethoxy-phenyl)-2-propenoic acid 4-(nitrooxy-methyl)phenyl ester;
(E)-3-(3,4-Dimethoxy-phenyl)-2-propenoic acid 3-(nitrooxy-methyl)phenyl amide;
(E)-3-(3,4-Dimethoxy-phenyl)-2-propenoic acid 2-(nitrooxy-methyl)phenyl amide;
(E)-3-(3,4-Dimethoxy-phenyl)-2-propenoic acid 4-(nitrooxy-methyl)phenyl amide;
(E)-3-(4-hydroxy-3-methoxy-phenyl)-2-propenoic acid 4-(nitrooxy)butyl ester;
(E)-3-(4-hydroxy-3-methoxy-phenyl)-2-propenoic acid 3-(nitrooxy-methyl)phenyl ester;
(E)-3-(4-hydroxy-3-methoxy-phenyl)-2-propenoic acid 6-(nitrooxy-methyl)-2-pyridinylmethylester hydrochloride;
(E)-3-(4-hydroxy-3-methoxy-phenyl)-2-propenoic acid 2-methoxy-4-[(1E)-3-[4-(nitrooxy)butoxy]-3-oxo-1-propenyl]phenyl ester;
(nitrooxy-methyl)phenyl-2-hydroxybenzoate;
(E)-3-(3,4-Dihydroxy-phenyl)-2-propenoic acid 3-(nitrooxy methyl)phenyl ester;
(E)-3-(3,4-Dihydroxy-phenyl)-2-propenoic acid 2-(nitrooxy methyl)phenyl ester;
(E)-3-(3,4-Dihydroxy-phenyl)-2-propenoic acid 4-(nitrooxy methyl)phenyl ester;
(E)-3-(3,4-Dihydroxy-phenyl)-2-propenoic acid 3-(nitrooxy methyl)phenyl amide;
(E)-3-(3,4-Dihydroxy-phenyl)-2-propenoic acid 2-(nitrooxy methyl)phenyl amide;
(E)-3-(3,4-Dihydroxy-phenyl)-2-propenoic acid 4-(nitrooxy methyl)phenyl amide;
(E)-3-(3,5-Dihydroxy-phenyl)-2-propenoic acid 3-(nitrooxy-methyl)phenyl ester;
(E)-3-(3,5-Dihydroxy-phenyl)-2-propenoic acid 3-(nitrooxy-methyl)phenyl amide;
(E)-3-(3-hydroxy-4-methoxy-phenyl)-2-propenoic acid 3-(nitrooxy-methyl)phenyl ester;
(E)-3-(3-hydroxy-4-methoxy-phenyl)-2-propenoic acid 3-(nitrooxy-methyl)phenyl amide;
(E)-3-(3,4-Dimethoxy-phenyl)-2-propenoic acid 3-(nitrooxy-methyl)phenyl ester;
(E)-3-(3,4-Dimethoxy-phenyl)-2-propenoic acid 2-(nitrooxy-methyl)phenyl ester;
(E)-3-(3,4-Dimethoxy-phenyl)-2-propenoic acid 4-(nitrooxy-methyl)phenyl ester;
(E)-3-(3,4-Dimethoxy-phenyl)-2-propenoic acid 3-(nitrooxy-methyl)phenyl amide;
(E)-3-(3,4-Dimethoxy-phenyl)-2-propenoic acid 2-(nitrooxy-methyl)phenyl amide;
(E)-3-(3,4-Dimethoxy-phenyl)-2-propenoic acid 4-(nitrooxy-methyl)phenyl amide;
(E)-3-(4-hydroxy-3-methoxy-phenyl)prop-2-enoic acid;
(E)-3-(4-hydroxy-3-methoxy-phenyl)-2-propenoic acid 4-(nitrooxy)butyl ester;
(E)-3-(4-hydroxy-3-methoxy-phenyl)-2-propenoic acid 3-(nitrooxy-methyl)phenyl ester;
(E)-3-(4-hydroxy-3-methoxy-phenyl)-2-propenoic acid 6-(nitrooxy-methyl)-2-pyridinylmethylester hydrochloride;
(E)-3-(4-hydroxy-3-methoxy-phenyl)-2-propenoic acid 2-methoxy-4-[(1E)-3-[4-(nitrooxy)butoxy]-3-oxo-1-propenyl]phenyl ester;
3-(nitrooxy-methyl)phenyl-2-hydroxybenzoate.

The preferred compounds of formula (I) of the present invention are:

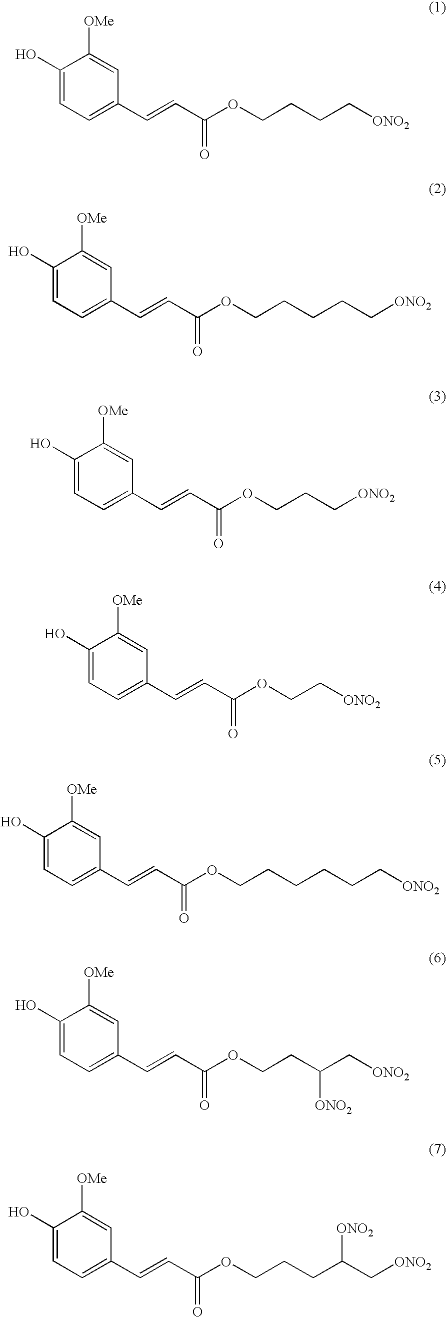

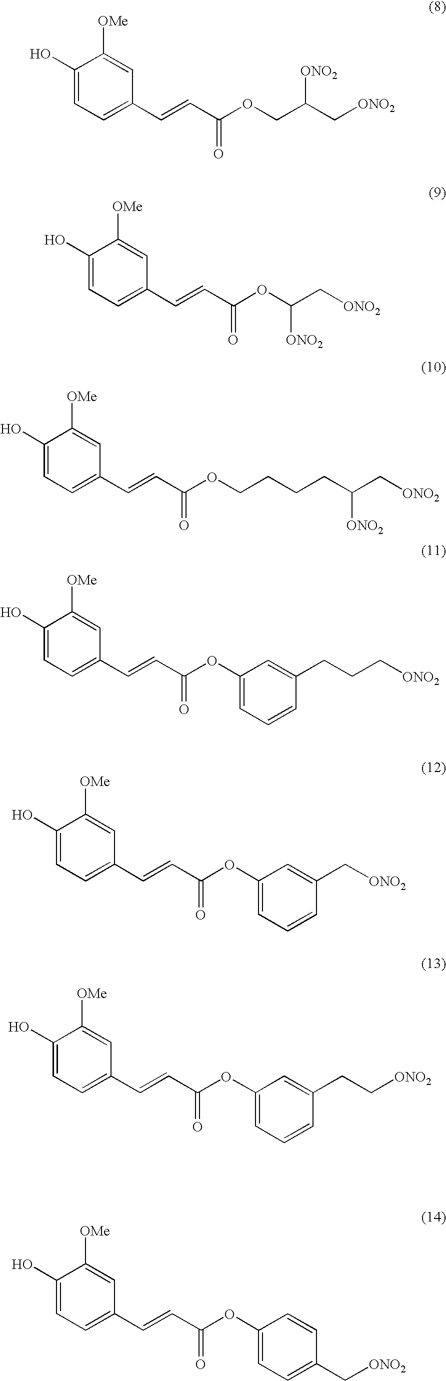

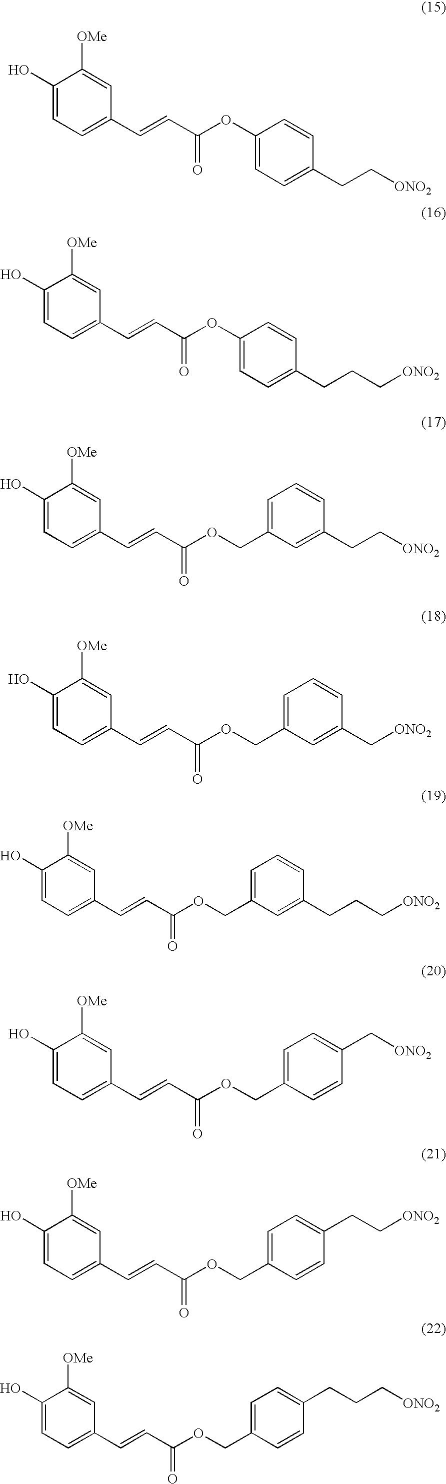

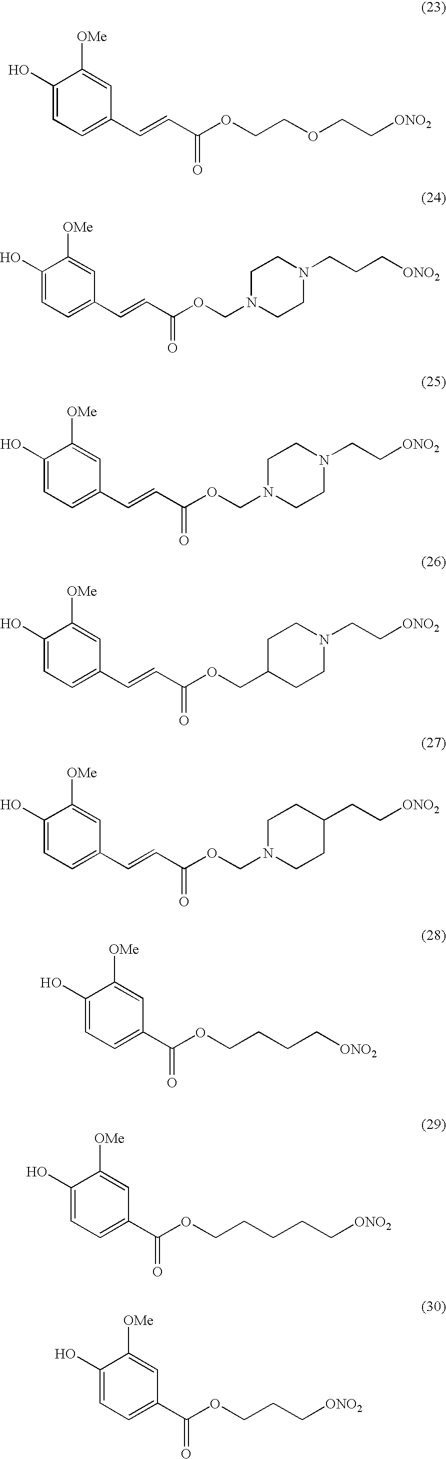

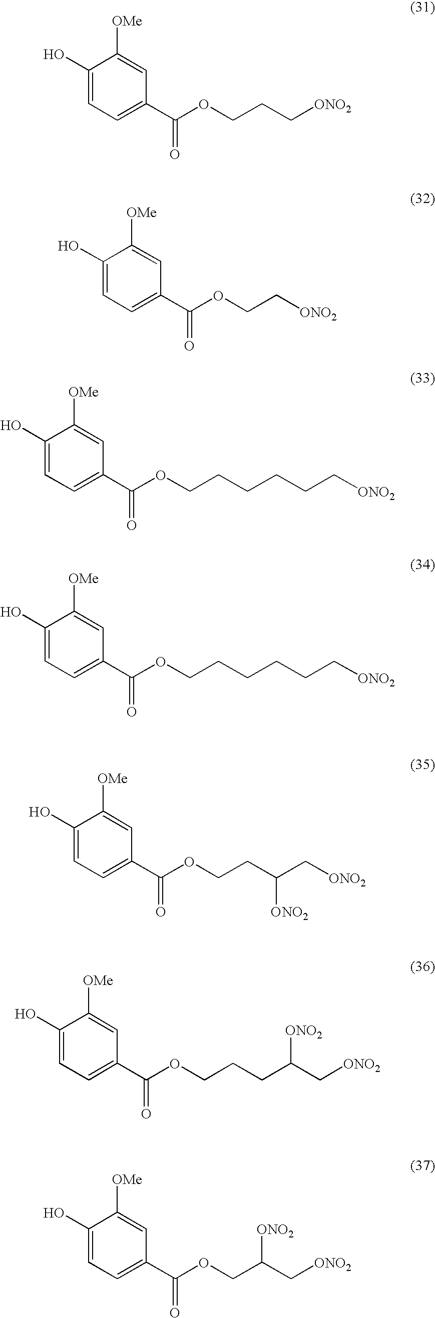

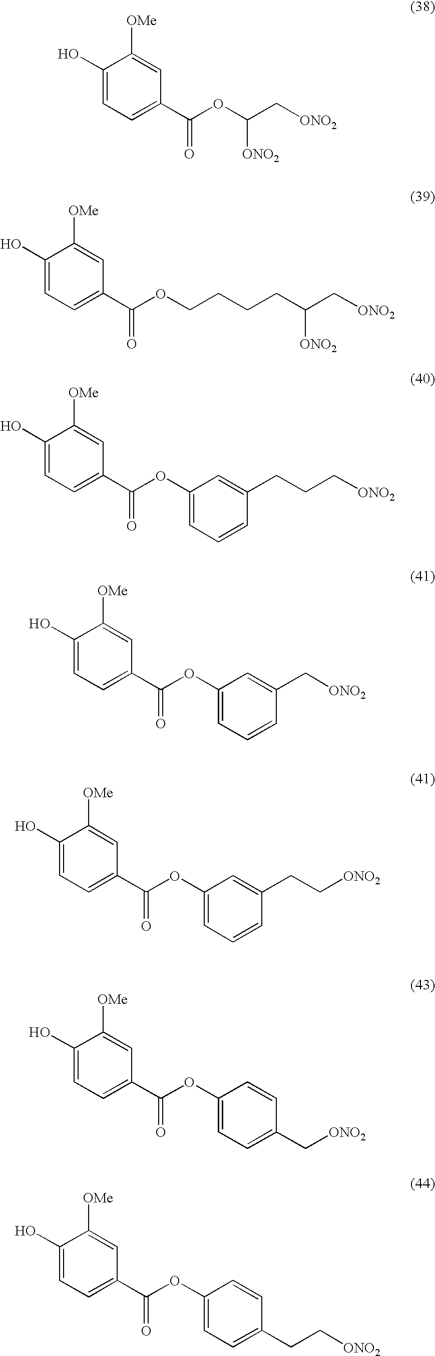

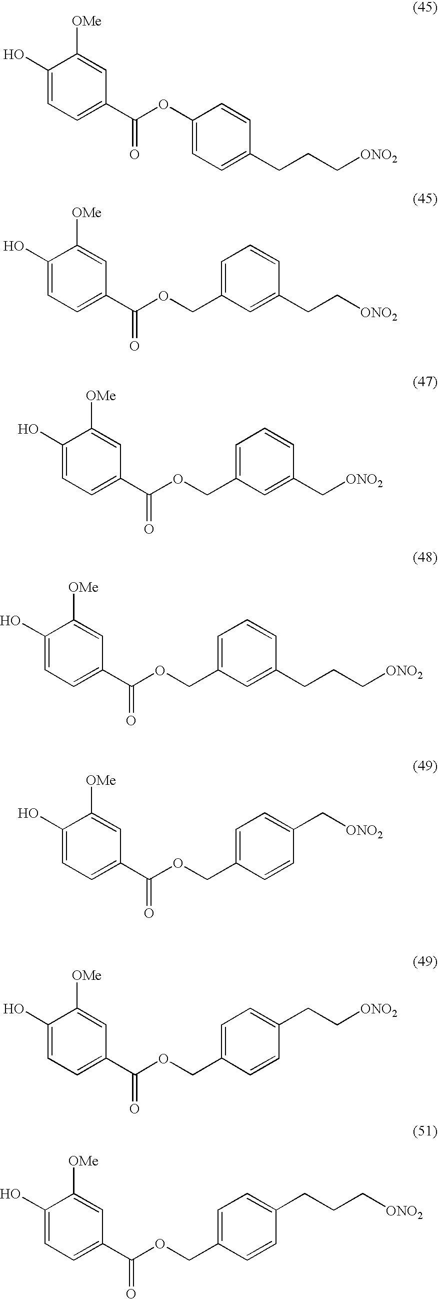

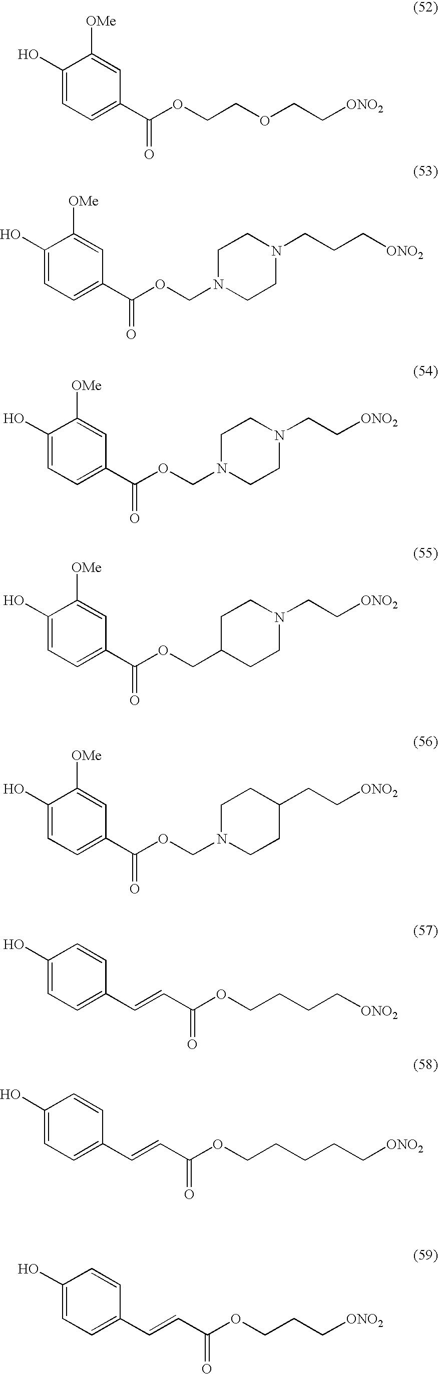

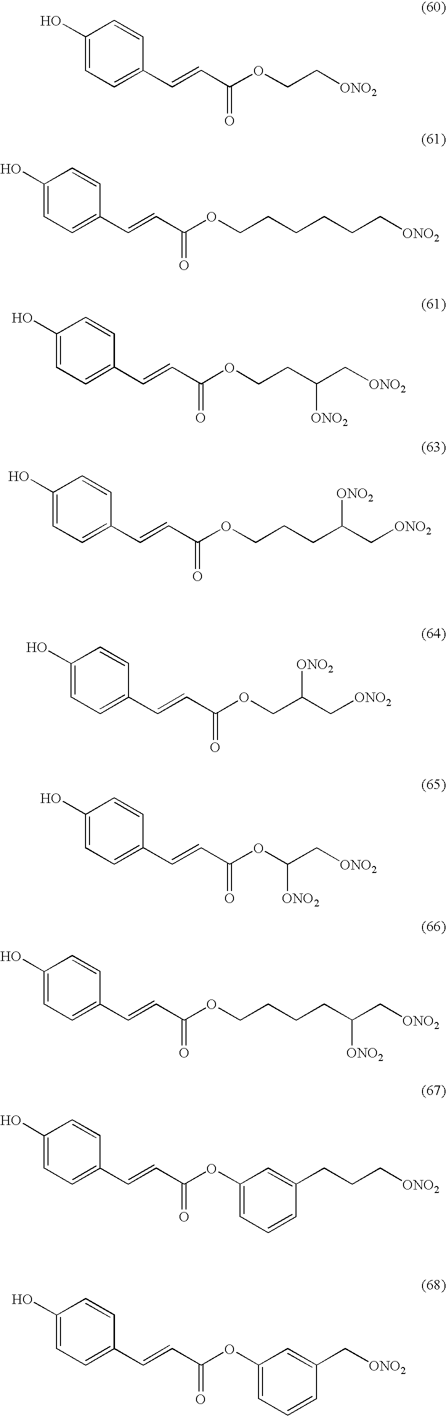

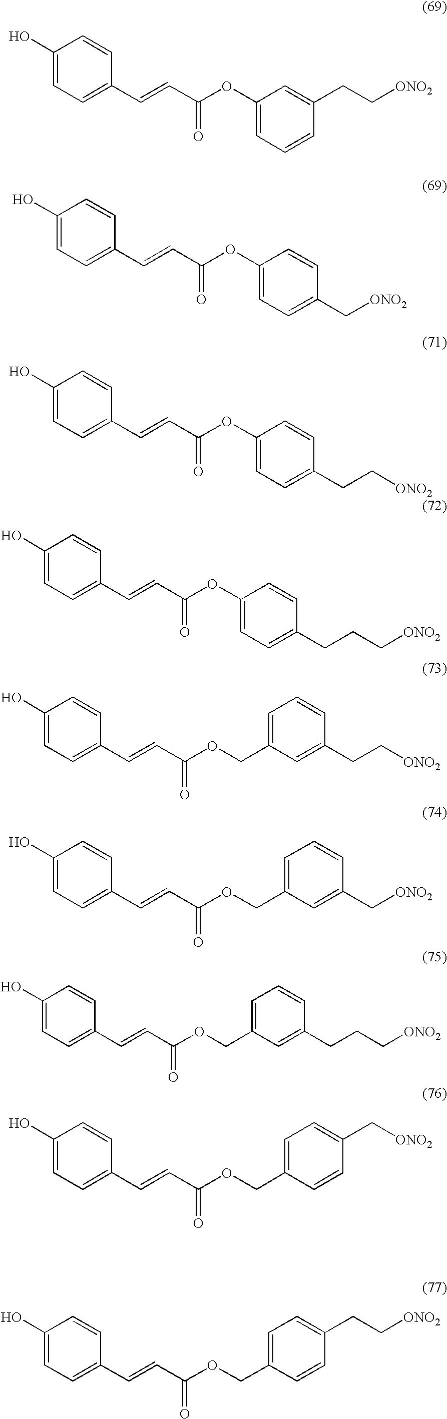

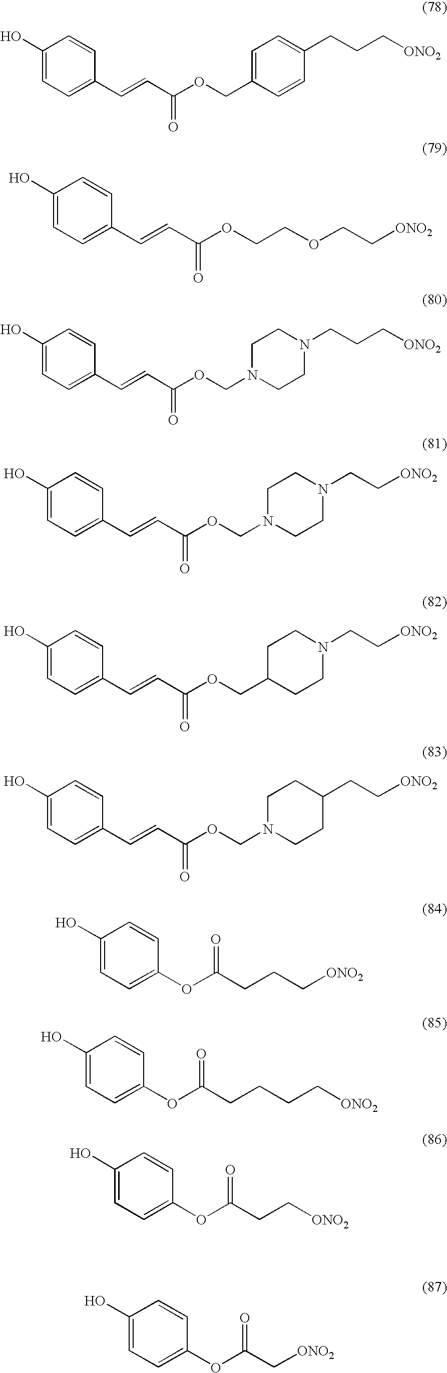

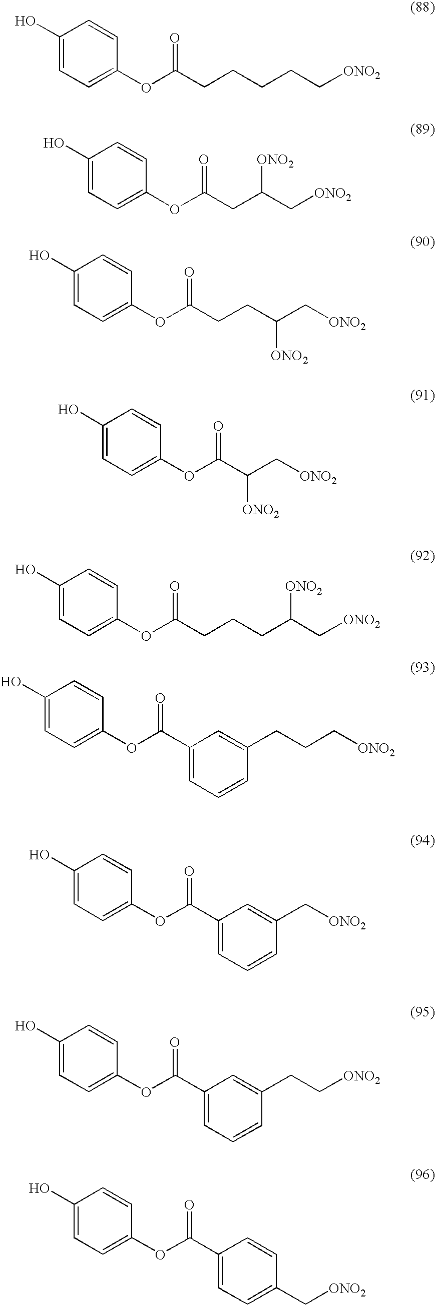

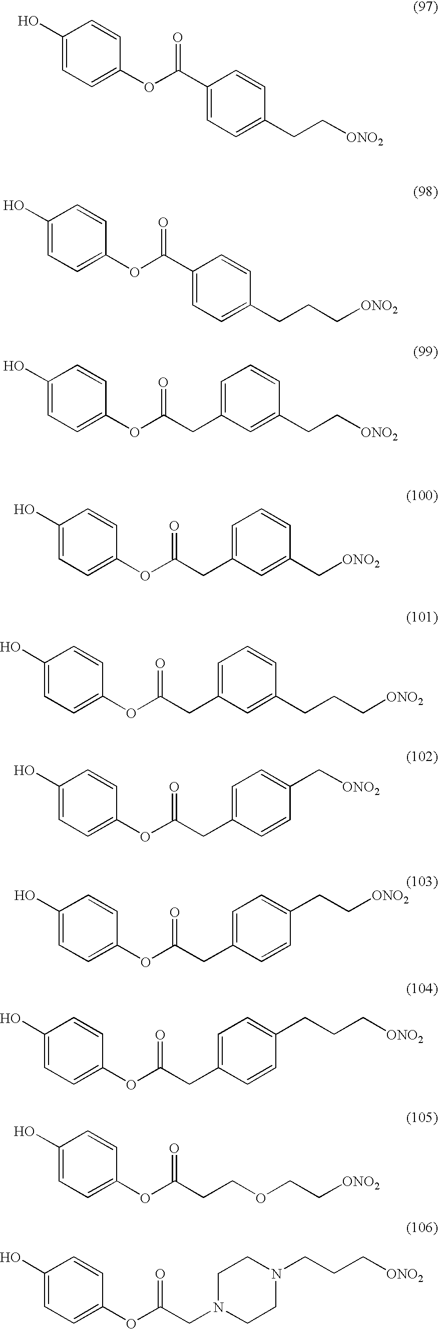

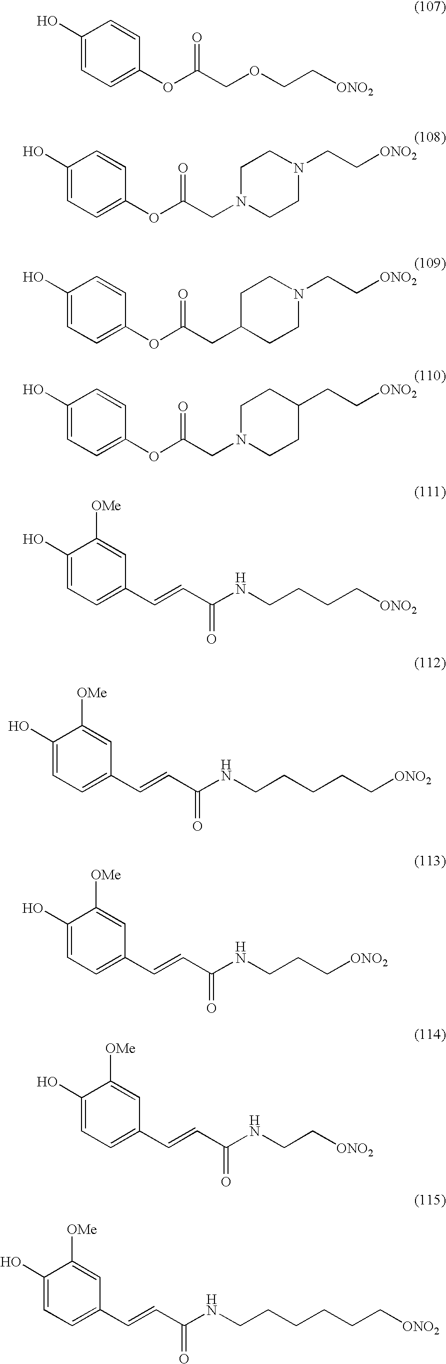

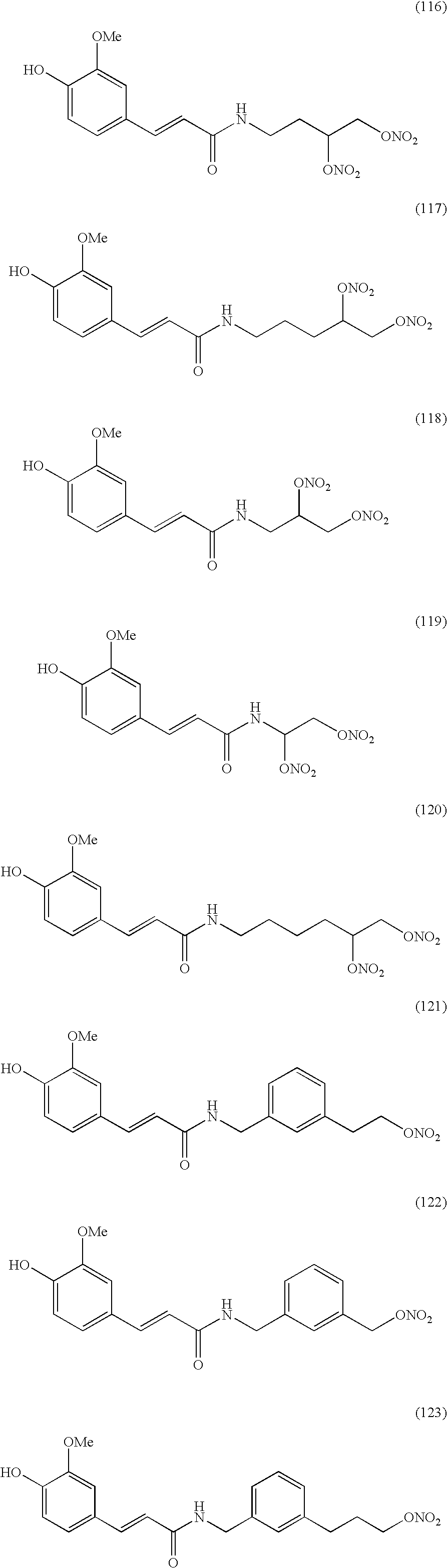

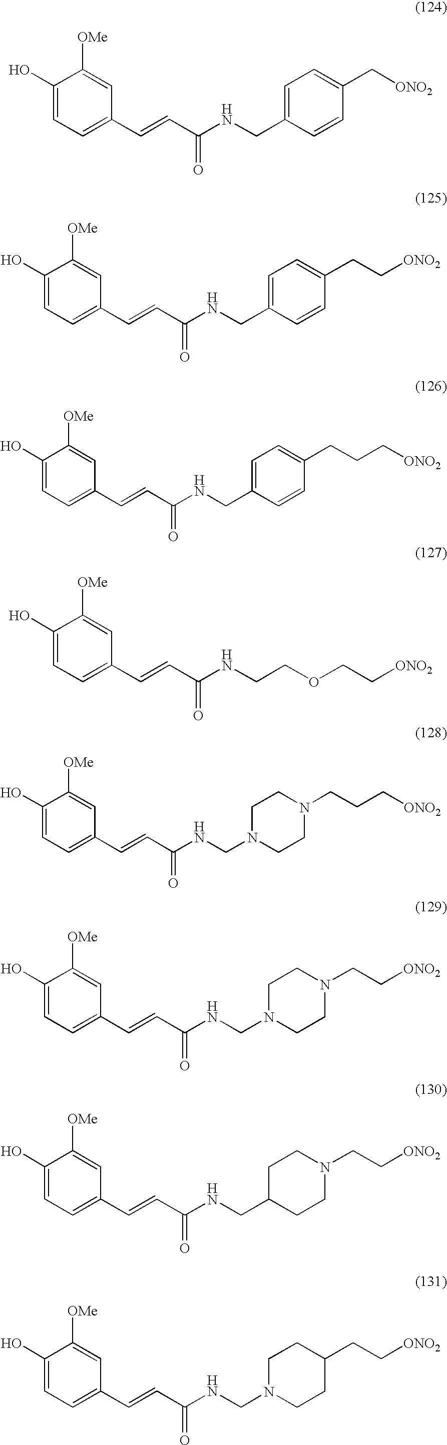

The compounds of general formula (I) of the present invention are synthesized by the synthetic procedures mentioned hereunder.

### EXPERIMENTAL PART

1) The compounds of general formula (I) as above defined wherein X is —C(O)—O, —C(O)—S, —C(O)—NR⁶ can be obtained:

i) by reacting a compound of formula (III)

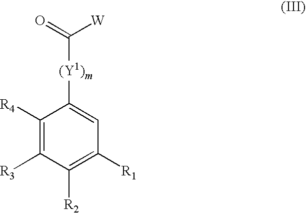

wherein
R₁, R₂, R₃, R₄, Y¹ and m are as above defined; W is —OH, Cl, or —OC(O)R⁸ wherein R⁸ is R⁶, as above defined, or Act, wherein Act is a carboxylic acid activating group used in peptide chemistry as:

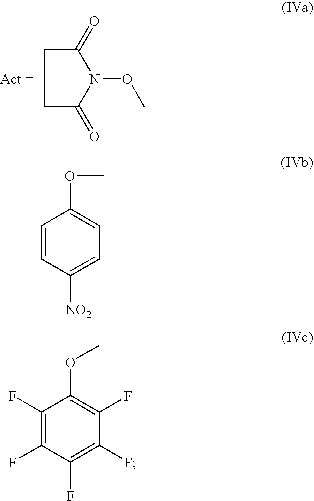

with a compound of formula (V) Z—Y-Q wherein Y is as above defined, Z is HX₁ or Z₁, wherein X₁ is O, S, NR⁸ wherein R⁸ is as above defined and Z₁ is selected from the group consisting of: chlorine, bromine, iodine, mesyl, tosyl;

### Q is —ONO₂ or Z₁ and

ii) when Q is Z₁, by converting the compound obtained in the step i) into nitro derivative by reaction with a nitrate source such as silver nitrate, lithium nitrate, sodium nitrate, potassium nitrate, magnesium nitrate, calcium nitrate, iron nitrate, zinc nitrate or tetraalkylammonium nitrate (wherein alkyl is C₁-C₁₀ alkyl) in a suitable organic solvent such as acetonitrile, tetrahydrofurane, methyl ethyl ketone, ethyl acetate, DMF, the reaction is carried out, in the dark, at a temperature from room temperature to the boiling temperature of the solvent. Preferred nitrate source is silver nitrate
The reaction of a compound of formula (III) wherein W═OH, R₁, R₂, R₃, R₄, Y¹ and m are as above defined, with a compound of formula (V) wherein Y and Q are as above defined, Z is HX₁ wherein X₁ is as above defined may be carried out in presence of a dehydrating agent as dicyclohexylcarbodiimide (DCC) or N′-(3-dimethylaminopropyl)-N-ethylcarbodiimide hydrochloride (EDAC) and a catalyst, such as N,N-dimethylamino pyridine (DMAP) or in presence of other known condensing reagents such as O-(7-azabenzotriazol-1-yl)-N,N,N′,N′-tetramethyluronium hexafluorophosphate (HATU).

The reaction is carried out in an inert organic dry solvent such as N,N′-dimethylformamide, tetrahydrofurane, benzene, toluene, dioxane or a polyhalogenated aliphatic hydrocarbon at a temperature from −20° C. to 40° C. The reaction is completed within a time range from 30 minutes to 36 hours.

The compounds of formula (III) wherein W═OH are commercially available.

The reaction of a compound of formula (III) wherein W=—OC(O)R⁸ wherein R⁸ is as above defined, with a compound of formula (V) wherein Y is as above defined, Z is —OH and Q is —ONO₂ may be carried out in presence of a catalyst, such as N,N-dimethylamino pyridine (DMAP). The reaction is carried out in an inert organic solvent such as N,N′-dimethylformamide, tetrahydrofurane, benzene, toluene, dioxane, a polyhalogenated aliphatic hydrocarbon at a temperature from −20° C. and 40° C. The reaction is completed within a time range from 30 minutes to 36 hours.

The compounds of formula (III) wherein W=—OC(O)R⁶ may be obtained from the corresponding acids wherein W═OH by reaction with a chloroformate such as isobutylchloroformate, ethylchloroformate in presence of a non-nucleophilic base such as triethylamine in an inert organic solvent such as N,N′-dimethylformamide, tetrahydrofurane or a polyhalogenated aliphatic hydrocarbon at a temperature from −20° C. and 40° C. The reaction is completed within a time range from 1 to 8 hours.

The reaction of a compound of formula (III) wherein W=Act with a compound of formula (V) wherein Y is as above defined, Z is —OH and Q is —ONO₂ may be carried out in presence of an inorganic or organic base in an aprotic polar/non-polar solvent such as DMF, THF or CH₂Cl₂ at temperatures in the range between 0° C. and 65° C. or in a double phase system H₂O/Et₂O at temperatures in the range between 20° C. and 40° C.; or in the presence of DMAP and a Lewis acid such as Sc(OTf)₃ or Bi(OTf)₃ in solvents such as DMF or CH₂Cl₂.

The compounds of formula (III) wherein W=—OC(O)Act may be obtained from the corresponding acids wherein W═OH as know in literature.

The reaction of a compound of formula (III) wherein W═OH with a compound of formula (V) wherein Y is as above defined, Z is Z₁ and Q is —ONO₂ may be carried out in presence of a organic base such as 1,8-diazabiciclo[5.4.0]undec-7-ene (DBU), N,N-diisopropylethylamine, diisopropylamine or inorganic base such as alkaline-earth metal carbonate or hydroxide, potassium carbonate, cesium carbonate, in an inert organic solvent such as N,N′-dimethylformamide, tetrahydrofuran, acetone, methyl ethyl ketone, acetonitrile or a polyhalogenated aliphatic hydrocarbon at a temperature from −20° C. to 40° C., preferably from 5° C. to 25° C. The reaction is completed within a time range from 1 to 8 hours. When Z₁ is chosen among chlorine or bromine the reaction is carried out in presence of a iodine compound such as KI.

The reaction of a compound of formula (III) wherein W═Cl with a compound of formula (V) wherein Y is as above defined, Z is —OH and Q is —ONO₂ may be carried out in presence of a of an organic base such as N,N-dimethylamino pyridine (DMAP), triethylamine, pyridine. The reaction is carried out in an inert organic solvent such as N,N′-dimethylformamide, tetrahydrofuran, benzene, toluene, dioxane or a polyhalogenated aliphatic hydrocarbon at a temperature from −20° C. to 40° C. The reaction is completed within a time range from 30 minutes to 36 hours.

The compounds of formula (III) wherein W═Cl may be obtained from the corresponding acids wherein W═OH by reaction with thionyl chloride, oxalyl chloride or halides of P^{III} or P^{V} in inert solvents such as toluene, chloroform or DMF.

The compounds of formula HO—Y—ONO₂, wherein Y is as above defined can be obtained as follows. The corresponding diol derivative, commercially available or synthesized as known in literature, is converted into HO—Y—Z₁, wherein Z₁ is as above defined, by known reactions, for example by reaction with thionyl chloride, oxalyl chloride, halides of P^{III} or P^{V}, mesyl chloride or tosyl chloride, in inert solvents such as toluene, chloroform, DMF, etc. The conversion to the nitro derivative is carried out as above described. Alternatively the diol derivative can be nitrated by reaction with nitric acid and acetic anhydride in a temperature range from −50° C. to 0° C. according to methods known in literature.

Alternatively the alcohol of formula HO—Y—ONO₂ wherein Y is a straight or branched C₁-C₂₀ alkyl substituted by a ONO₂ group having the following formula —Y²—(CHONO₂)—CH₂— can be obtained from the alcohol of formula HO—Y²—CH═CH₂ by treatment with iodine and silver nitrate in acetonitrile at a temperature between −20° C. and 80° C.

The compounds of formula Z₁—Y—ONO₂, wherein Y and Z₁ are as above defined can be obtained from the halogen derivative Z₁—Y-Hal, commercially available or synthesized according to methods well known in literature, by conversion to the nitro derivative as above described.

The compounds of formula H—X—Y—Z₁, wherein X, Y and Z₁ are as above defined can be obtained from the hydroxyl derivative H—X—Y—OH, commercially available or synthesized according to methods well known in literature, by known reactions, for example by reaction with thionyl chloride, oxalyl chloride, halides of P^{III} or P^{V}, mesyl chloride or tosyl chloride in inert solvents such as toluene, chloroform, DMF, etc.

2) The compound of general formula (I) as above defined wherein X is —C(O)O— and Y is alkyl C₄ can be obtained:

i) by reacting a compound of formula (VI)

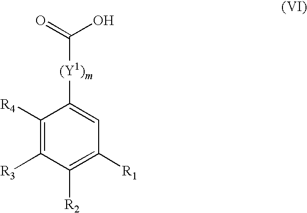

wherein R₁, R₂, R₃, R₄, Y¹ and m are as above defined, with tetrahydrofurane in presence of triphenylphosphine and tetrabromomethane at a temperature from −20° C. to 40° C. The reaction is completed within a time range from 30 minutes to 36 hours and
ii) by converting the compound obtained in the step i) into nitro derivative by reaction with a nitrate source as above described.

3) The compounds of general formula (I) as above defined wherein X is —OC(O)— can be obtained:

i) by reacting a compound of formula (VII)

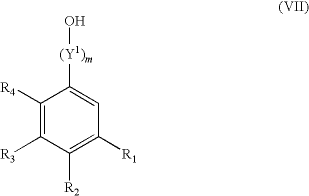

wherein
R₁, R₂, R₃, R₄, Y¹ and m are as above defined, with a compound of formula (VIII) W—C(O)—Y-Q wherein Y, Q and W are as above defined, and
ii) when Q is Z₁, by converting the compound obtained in the step i) into nitro derivative by reaction with a nitrate source as above described.

The reaction of a compound of formula (VII) wherein R₁, R₂, R₃, R₄, Y¹ and m are as above defined, with a compound of formula (VIII) wherein Y, Q and W are as above defined, may be carried out as above described in 1).

The compounds of formula W—C(O)—Y—ONO₂ wherein Y is as above defined and W═OH can be obtained as follows. The corresponding alcohol derivative, commercially available, or synthesized by well known reactions, is converted to HO—C(O)—Y—Z₁, wherein Z₁ is as above defined, by known reactions, for example by reaction with thionyl chloride, oxalyl chloride, halides of P^{III} or P^{V}, mesyl chloride or tosyl chloride in inert solvents such as toluene, chloroform, DMF, etc. The conversion to the nitro derivative is carried out as above described. Alternatively the alcohol derivative can be nitrated by reaction with nitric acid and acetic anhydride in a temperature range from −50° C. to 0° C. according to methods well known in literature.

Alternatively the acids of formula HO—C(O)—Y—ONO₂ wherein Y is a straight or branched C₁-C₂₀ alkyl substituted by a —ONO₂ group having the following formula —Y²—(CHONO₂)—CH₂— can be obtained from the acid of formula HO—C(O)—Y²—CH═CH₂ by treatment with iodine and silver nitrate in acetonitrile at a temperature between −20° C. and 80° C.

The compounds of formula W—C(O)—Y—ONO₂ wherein Y is as above defined and W=Act can be obtained from the corresponding acid as known in literature.

The compounds of formula W—C(O)—Y—ONO₂ wherein Y is as above defined and W═Cl can be obtained from the corresponding acid as known in literature.

4) The compounds of general formula (I) as above defined wherein X is —OC(O)O— can be obtained:

i) by reacting a compound of formula (VII) with a compound of formula (IX) Act₁-C(O)—O—Y-Q wherein Y, Q are as above defined and Act is as defined in (IVb) and (IVc), and
ii) when Q is Z₁, by converting the compound obtained in the step i) into nitro derivative by reaction with a nitrate source as above described.

The reaction of a compound of formula (VII) wherein R₁, R₂, R₃, R₄, Y¹ and m are as above defined, with a compound of formula (IX) wherein Y, Q and Act are as above defined, may be carried out as above described in 1).

The compounds of formula Act₁-C(O)—O—Y—ONO₂ wherein Y and Act are as above defined can be obtained from the corresponding alcohol as known in literature.

### Example 1

### Synthesis of Ferulic Acid 4-(nitrooxy)butyl ester

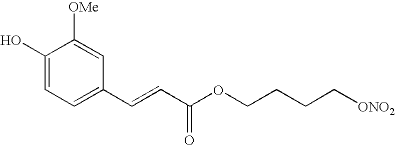

### A) Ferulic acid 4-(bromo)butyl ester

To a solution of ferulic acid (1 g, 5.15 mmol) in tetrahydrofurane (40 ml), triphenylphosphine (2.7 g, 10.3 mmol) and tetrabromomethane (3.41 g, 10.3 mmol) were added. The mixture was stirred at room temperature for 4 hours. The mixture was filtered and the solvent was evaporated under vacuum. The crude residue was purified by silica gel chromatography, eluent n-hexane/ethyl acetate 7/3. The product (0.77 g) was obtained as a yellow solid. (Yield 46%). M.p.=83-88° C.

### B) Ferulic acid 4-(nitrooxy)butyl ester

A solution of compound A (0.8 g, 2.43 mmol) and silver nitrate (1.2 g, 7.29 mmol) in acetonitrile (50 ml) was stirred at 40° C., in the dark, for 16 hours. The precipitated (silver salts) was filtered off and the solvent was evaporated under vacuum. The residue was purified by flash chromatography, eluent n-hexane/ethyl acetate 75/25. The product (0.4 g) was obtained as white powder (yield 53%). M.p.=63-64° C.

### Example 2

### Synthesis of Ferulic Acid 2-(nitrooxy)ethyl ester

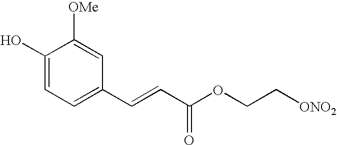

### C) Ferulic acid 2-bromoethyl ester

To a solution of ferulic acid (2 g, 10.3 mmol) in chloroform (50 ml) 2-bromoethanol (4.36 ml, 61.8 mmol) and DMAP (cat. amount) were added. The reaction was cooled at 0° C. and EDAC (2.96 g, 15.45 mmol) was added. The reaction was stirred at room temperature for 6 hours. The solution was treated with water and chloroform, the organic layers were dried with sodium sulfate and concentrated under reduced pressure. The residue was purified by flash chromatography, (Biotage Horizon System), eluent n-hexane/ethyl acetate 72/28 The product (1.73 g) was obtained (yield 55.8%).

### D) Ferulic Acid 2-(nitrooxy)ethyl ester

A solution of compound C (0.240 g, 0.8 mmol) and silver nitrate (0.16 g, 0.96 mmol) in acetonitrile (15 ml) was heated at 120° C. for 240 sec. with Microwave Emrys™ Creator (Personal Chemistry). The precipitated (silver salts) was filtered off and the solvent was evaporated under vacuum. The residue was purified by flash chromatography (Biotage Horizon System), eluent n-hexane/ethyl acetate 75/25. The product (0.127 g) was obtained as white powder (yield 56.4%)

### Example 3

### Synthesis of Vanillic Acid 4-(nitrooxy)butyl ester

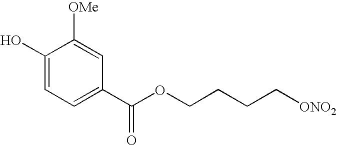

### E) Vanillic acid 4-(bromo)butyl ester

To a solution of vanillic acid (1.5 g, 8.92 mmol) in tetrahydrofurane (100 ml), triphenylphosphine (4.7 g, 17.8 mmol) and tetrabromomethane (5.92 g, 17.8 mmol) were added. The mixture was stirred at room temperature for 4 hours, then filtered and the solvent was evaporated under vacuum. The crude residue was purified by silica gel chromatography, eluent n-hexane/ethyl acetate 8/2. The product (2.44 g) was obtained as a solid. (Yield 90.4%). M.p.=83-88° C.

### F) Vanillic Acid 4-(nitrooxy)butyl ester

A solution of compound E (0.285 g, 0.94 mmol) and silver nitrate (0.4 g, 2.3 mmol) in acetonitrile (15 ml) was heated at 115° C. for 900 sec. with Microwave Emrys™ Creator (Personal Chemistry). The precipitated (silver salts) was filtered off and the solvent was evaporated under vacuum. The residue was purified by flash chromatography, eluent n-hexane/ethyl acetate 75/25. The product (0.117 g) was obtained as white powder (yield 43.7%)

### Pharmacological Examples

The efficacy of nitrooxyderivatives of antioxidant compounds of formula (I) in the treatment of neuropathic pain was evaluated in the model of hyperalgesic responses following chronic constriction injuries of the rat sciatic nerve. The pharmacological activity of the compounds of formula (I) was compared to that of the known analgesic drugs. The parent not-derivatized antioxidant drug was used as reference. The efficacy of the equimolar combination of compounds of formula (I) and analgesic drugs selected between the group of gabapentin, tiagabine and pregabalin was also evaluated, on the same experimental model.

### Example F1

### Effects of Vehicle, Ferulic Acid 4-(nitrooxy)butyl ester, Ferulic Acid and Gabapentin on Hyperalgesic Responses Following Chronic Constriction Injuries

### Chronic Constriction Injury (CCI)-Induced Mechanical Hyperalgesia in Rats.

The method described by Bennet and Xie (1988) was generally followed. Rats were anesthetized with chloral hydrate (380 mg/kg ip, Sigma). The right common sciatic nerve was exposed at the level of the middle of the high by blunt dissection through the biceps femoris. Proximal to the sciatic's trifurcation, about 12 mm of nerve was freed of adhering tissue and four ligatures (3/0 silk suture) were tied loosely around it with about 1 mm spacings. Ligatures were tied such that the diameter of the nerve was only barely constricted. The desired degree of constriction retarded, but did not arrest, circulation through the superficial epineural vasculature. The incision was closed in layers. The experiments were then carried out 1 week after surgery. Mechanical hyperalgesia was determined with an analgesimeter (Ugo Basile, Italy), using the modified Randall-Selitto method. Briefly, a conical stylus with a hemispherical tip was places upon the middle of hind paw dorsum. The animal was gently restrained and calibrated pressure of gradually increasing intensity was applied until the rat withdrew the hindpaw. Threshold pressure (g) of both hindpaws was determined every 15 minutes, with two measurements before the treatments (pretest), and from 15 to 60 minutes after treatment. An arbitrary cut-off value of 240 g was adopted.

### Results I

Seven days after sciatic nerve ligation, the mechanical paw withdrawal threshold (PWT) of the CCI-lesioned ipsilateral paw (ipsi) was stable and significantly lower than that of the respective contralateral unlesioned paw (contra). As reported in table I, the administration of ferulic acid 4-(nitrooxy)butyl ester significantly increased ipsi PWT to that observed following the administration of gabapentin at equal dose. Ferulic acid 4-(nitrooxy)butyl ester also enhanced PWT contralateral to the lesion side and differently from gabapentin did not induce any appreciable sedative effect. The effects of ferulic acid 4-(nitrooxy)butyl ester were also greater than that of ferulic acid.

TABLE I

| | Ipsi-PWT_{30 min} | Contra-PWT_{30 min} |
|---|---|---|
| COMPOUND | (g) | (g) |
| Vehicle | 41.4 ± 3.3 | 78.5 ± 1.8 |
| Ferulic acid 4- | 76.3 ± 4.8* | 98.5 ± 4.8* |
| (nitrooxy)butyl ester | | |
| (175 μmol/kg, sc) | | |
| Gabapentin | 75.1 ± 9.8* | 84.3 ± 3.9 |
| (175 μmol/kg, sc) | | |
| Ferulic acid | 37.9 ± 15.5 | 71.4 ± 11.1 |
| (175 μmol/kg, sc) | | |

PWT = Paw Withdrawal Threshold;
sc = subcutaneous;
*p < 0.05 vs vehicle or ferulic acid

### Example F2

### Effects of Vehicle, Ferulic Acid 4-(nitrooxy)butyl ester or Gabapentin Alone or in Combination on Hyperalgesic Responses Following Chronic Constriction Injuries

### Results II

Table II shows the effects of ferulic acid 4-(nitrooxy)butyl ester, gabapentin and their combination. The administration of ferulic acid 4-(nitrooxy)butyl ester combined with gabapentin elicited greater effects as compared to gabapentin or ferulic acid 4-(nitrooxy)butyl ester administered alone. The combination elicited much longer response as compared to either drug alone being still fully effective 60 min after the administration, time at which the effects of individual drugs had completely disappeared.

TABLE II

| COMPOUND | PWT_{30 min} (g) | PWT_{60 min} (g) |
|---|---|---|
| Vehicle | 41.4 ± 3.3 | 34.3 ± 3.1 |
| Ferulic acid 4-(nitrooxy)butyl | 76.3 ± 4.8* | 37.5 ± 5.9 |
| ester (175 μmol/kg, sc) | | |
| Gabapentin (175 μmol/kg, sc) | 75.1 ± 9.8* | 37.6 ± 7.0 |
| Ferulic acid 4-(nitrooxy)butyl | 82.5 ± 1.9* | 70.4 ± 2.8# |
| ester (175 μmol/kg, sc) + | | |
| Gabapentin (175 μmol/kg, sc) | | |

PWT = Paw Withdrawal Threshold;
sc = subcutaneous;
*p < 0.05 vs respective vehicle;
#p < 0.05 vs other groups

1. Use of compound of formula (I) for the preparation of medicament for the treatment of chronic neuropathic pain 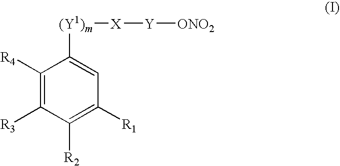
or pharmaceutically acceptable salts or stereoisomers thereof, wherein in formula (I)
m is an integer equal to 0 or 1;
Y¹ is —CH═CH—(CH₂)ₘ¹, wherein m¹ is an integer from 0 to 3, or —(CH₂)ₘ²— wherein m² is an integer from 1 to 3;
R₁, R₂, R₃, R₄ are independently selected from H, OH, —OR₅ wherein R₅ is a straight or branched (C₁-C₁₀)-alkyl, straight or branched C₁-C₂₀ alkyl, with the proviso that at least one of R₁, R₂, R₃, R₄ is not H;
X is —OC(O)—, —OC(O)O—, —C(O)O—, —C(O)NR₆—, —C(O)S— wherein R₆ is H or a (C₁-C₅)-alkyl;
Y is a bivalent radical having the following meaning:
c) straight or branched C₁-C₂₀ alkylene optionally substituted with one or more substituents independently selected from halogen atoms, hydroxy, —ONO₂ or T, wherein T is —OC(O)(C₁-C₁₀ alkyl)-ONO₂ or —O(C₁-C₁₀ alkyl)-ONO₂; cycloalkylene with 5 to 7 carbon atoms into cycloalkylene ring, the ring being optionally substituted with side chains T₁, wherein T₁ is straight or branched C₁-C₁₀ alkyl; 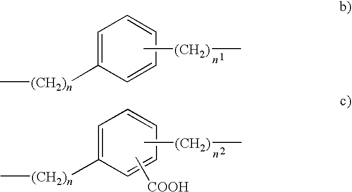
wherein n is an integer from 0 to 20, preferably n is an integer from 0 to 5,
n¹ an integer from 1 to 20, preferably n¹ is an integer from 1 to 5; 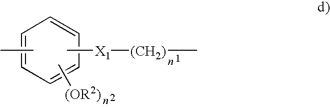
wherein
X₁ is —OC(O)— or —C(O)O—, n² is an integer from 1 to 3 and R₂ is H or CH₃;
n¹ is as defined above and n² is an integer from 0 to 2; 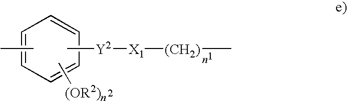
wherein:
Y² is —CH₂—CH₂—(CH₂)ₙ²—; or —CH═CH—(CH₂)ₙ²—;
X₁ is —OC(O)— or —C(O)O—, n² is an integer from 1 to 3 and R₂ is H or CH₃;
when Y is one of the bivalent radicals b) to e), the —ONO₂ group is bound to the —(CH₂)ₙ¹— group;
when Y is one of the bivalent radicals mentioned under b) to e), m is 1; 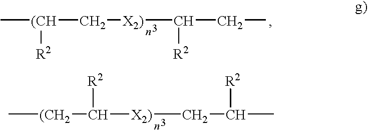
wherein X₂ is —O— or —S— or NR₆— wherein R₆ is as above defined, preferably X₂ is —O—, n³ is an integer from 1 to 6, preferably from 1 to 4, R² is as defined above, preferably R² is H; 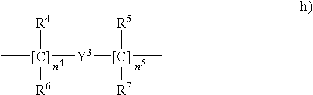
wherein:
n⁴ is an integer from 0 to 10, preferably n⁴ is 1;
n⁵ is an integer from 1 to 10, preferably n⁵ is an integer from 1 to 5;
R⁴, R⁵, R⁶, R⁷ are the same or different, and are H or straight or branched C₁-C₄ alkyl, preferably R⁴, R⁵, R⁶, R⁷ are H; wherein the —ONO₂ group is linked to 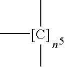
wherein n⁵ is as defined above;
Y³ is an heterocyclic saturated, unsaturated or aromatic 5 or 6 members ring, containing one or more heteroatoms selected from nitrogen, oxygen, sulfur, and is selected from

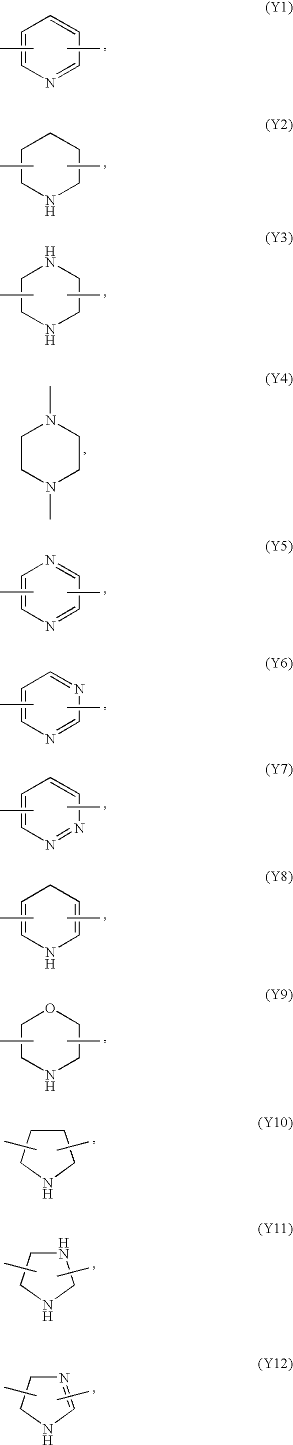

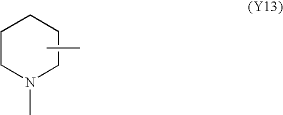

2. Use of a compound of formula (I) according to claim 1 wherein in formula (I)
R¹ is —OCH₃, R² is OH, R₃ and R₄ are H, m is 0 or m is 1 and Y¹ is —CH═CH—(CH₂)ₘ¹,
wherein m¹ is 0, or
R₁ and R₂ are OH, R₃ and R₄ are H, m is 0 or m is 1 and Y¹ is —CH═CH—(CH₂)ₘ¹,
wherein m¹ is 0, or Y¹ is —(CH₂)ₘ²— wherein m² is 2; or
R₁, R₃ and R₄ are H and R₂ is OH, m is 0 or m is 1 and Y¹ is —CH═CH—(CH₂)ₘ¹,
wherein m¹ is 0 or, or Y¹ is —(CH₂)ₘ²— wherein m² is 2;
R₁, R₂ and R₃ are OH, R₄ is H and m is 0
R₁ and R₃ are —OCH₃, R₂ is OH and R₄ is H, m is 0;
R₁ is OH, R₂ is —OCH₃, R₃ and R₄ are H and m is 0;
R₁ is OH, R₂ and R₃ are H, R₄ is —OCH₃, m is 0;
R₁ and R₄ are OH, R₂ and R₃ are H, m is 0
R₁ and R₃ are H and R₂ and R₄ are OH, m is 0.

3. Use of a compound of formula (I) according to claim 1 wherein in formula (I)
Y is selected from:
a) (C₁-C₁₀) alkylene or a (C₁-C₁₀) alkylene substituted with a —ONO₂; 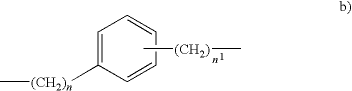
wherein n is 0 or 1, n¹ is an integer from 1 to 5; 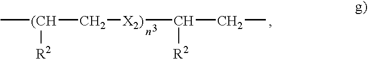
wherein X₂ is —O—, n³ is 1 and R² is H; 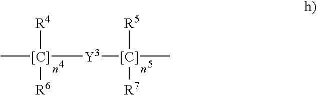
wherein:
n⁴ is n⁵ is an integer from 1 to r;
R⁴, R⁵, R⁶, R⁷ are H;
Y³ is (Y4) or (Y13);

4. Use according to claim 1 wherein the compound of formula (I) is ferulic acid 4-(nitrooxy)butyl ester.

5. A combination comprising a compound of formula (I) as described in claims 1 and an analgesic drug selected from the group of gabapentin, pregabalin and tiagabine.

6. A combination according to claim 5 comprising ferulic acid 4-(nitrooxy)butyl ester and gabapentin.

7. Use of a combination of claim 5 for the preparation of a medicament for treating chronic neuropathic pain.

8. Pharmaceutical composition comprising a combination according to claim 5 and pharmaceutical acceptable excipients.

9. A compound of formula (I) 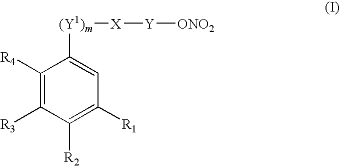
or pharmaceutically acceptable salts or stereoisomers thereof, wherein in formula (I)
m is an integer equal to 0 or 1;
Y¹ is —CH═CH—(CH₂)ₘ¹, wherein m¹ is an integer from 0 to 3, or —(CH₂)ₘ²— wherein m² is an integer from 1 to 3;
R₁, R₂, R₃, R₄ are independently selected from H, OH, —OR₅ wherein R₅ is a straight or branched (C₁-C₁₀)-alkyl, straight or branched C₁-C₂₀ alkyl, with the proviso that at least one of R₁, R₂, R₃, R₄ is not H;
X is —OC(O)—, —OC(O)O—, —C(O)O—, —C(O)NR₆—, —C(O)S— wherein R₆ is H or a (C₁-C₅)-alkyl;
Y is a bivalent radical having the following meaning:
d) straight or branched C₁-C₂₀ alkylene optionally substituted with one or more substituents independently selected from halogen atoms, hydroxy, —ONO₂ or T, wherein T is —OC(O)(C₁-C₁₀ alkyl)-ONO₂ or —O(C₁-C₁₀ alkyl)-ONO₂;
cycloalkylene with 5 to 7 carbon atoms into cycloalkylene ring, the ring being optionally substituted with side chains T₁, wherein T₁ is straight or branched C₁-C₁₀ alkyl; 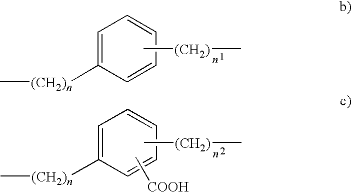
wherein n is an integer from 0 to 20, and n¹ is an integer from 1 to 20; 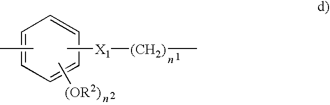
wherein
X₁ is —O0(O)— or —C(O)O—, n² is an integer from 1 to 3 and R² is H or CH₃;
n¹ is as defined above and n² is an integer from 0 to 2; 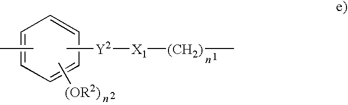
wherein:
Y² is —CH₂—CH₂—(CH₂)ₙ²—; or —CH═CH—(CH₂)ₙ²—;
X₁ is —OC(O)— or —C(O)O—, n² is an integer from 1 to 3 and R² is H or CH₃;
when Y is one of the bivalent radicals b) to e), —ONO₂ group is bound to —(CH₂)ₙ¹— group;
when Y is one of the bivalent radicals mentioned under b) to e), then m is 1; 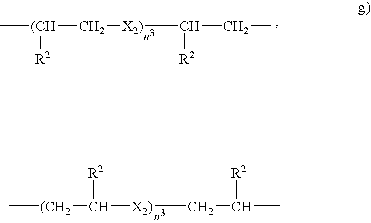
wherein X₂ is —O— or —S— or NR₆— wherein R₆ is as above defined, n³ is an integer from 1 to 6; 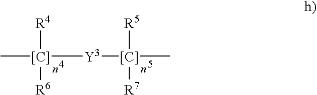
wherein:
n⁴ is an integer from 0 to 10;
n⁵ is an integer from 1 to 10;
R⁴, R⁵, R⁶, R⁷ are the same or different, and are H or straight or branched C₁-C₄ alkyl;
wherein the —ONO₂ group is linked to 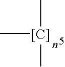
wherein n⁵ is as defined above;
Y³ is an heterocyclic saturated, unsaturated or aromatic 5 or 6 members ring, containing one or more heteroatoms selected from nitrogen, oxygen, sulfur, and is selected from 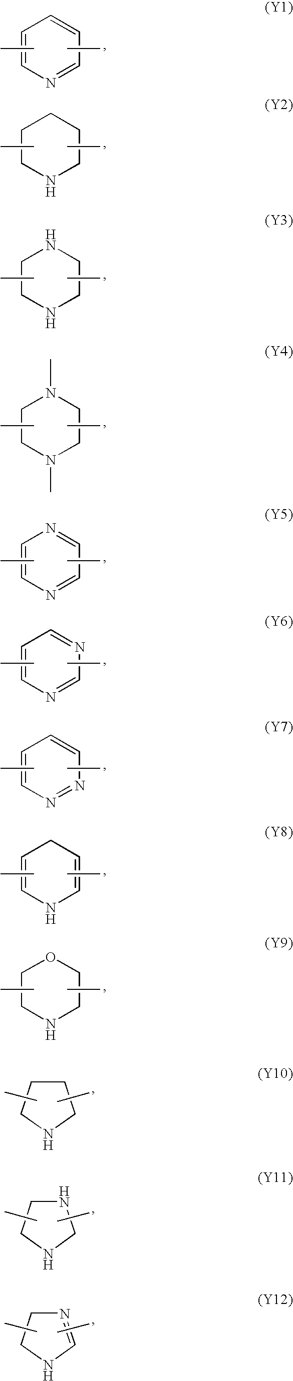 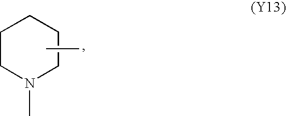
excluding:
(E)-3-(3,4-Dihydroxy-phenyl)-2-propenoic acid 3-(nitrooxy-methyl)phenyl ester;
(E)-3-(3,4-Dihydroxy-phenyl)-2-propenoic acid 2-(nitrooxy-methyl)phenyl ester;
(E)-3-(3,4-Dihydroxy-phenyl)-2-propenoic acid 4-(nitrooxy-methyl)phenyl ester;
(E)-3-(3,4-Dihydroxy-phenyl)-2-propenoic acid 3-(nitrooxy-methyl)phenyl amide;
(E)-3-(3,4-Dihydroxy-phenyl)-2-propenoic acid 2-(nitrooxy-methyl)phenyl amide;
(E)-3-(3,4-Dihydroxy-phenyl)-2-propenoic acid 4-(nitrooxy-methyl)phenyl amide;
(E)-3-(3,5-Dihydroxy-phenyl)-2-propenoic acid 3-(nitrooxy-methyl)phenyl ester;
(E)-3-(3,5-Dihydroxy-phenyl)-2-propenoic acid 3-(nitrooxy-methyl)phenyl amide;
(E)-3-(3-hydroxy-4-methoxy-phenyl)-2-propenoic acid 3-(nitrooxy-methyl)phenyl ester;
(E)-3-(3-hydroxy-4-methoxy-phenyl)-2-propenoic acid 3-(nitrooxy-methyl)phenyl amide;
(E)-3-(3,4-Dimethoxy-phenyl)-2-propenoic acid 3-(nitrooxy-methyl)phenyl ester;
(E)-3-(3,4-Dimethoxy-phenyl)-2-propenoic acid 2-(nitrooxy-methyl)phenyl ester;
(E)-3-(3,4-Dimethoxy-phenyl)-2-propenoic acid 4-(nitrooxy-methyl)phenyl ester;
(E)-3-(3,4-Dimethoxy-phenyl)-2-propenoic acid 3-(nitrooxy-methyl)phenyl amide;
(E)-3-(3,4-Dimethoxy-phenyl)-2-propenoic acid 2-(nitrooxy-methyl)phenyl amide;
(E)-3-(3,4-Dimethoxy-phenyl)-2-propenoic acid 4-(nitrooxy-methyl)phenyl amide;
(E)-3-(4-hydroxy-3-methoxy-phenyl)-2-propenoic acid 4-(nitrooxy)butyl ester;
(E)-3-(4-hydroxy-3-methoxy-phenyl)-2-propenoic acid 3-(nitrooxy-methyl)phenyl ester;
(E)-3-(4-hydroxy-3-methoxy-phenyl)-2-propenoic acid 6-(nitrooxy-methyl)-2-pyridinylmethylester hydrochloride;
(E)-3-(4-hydroxy-3-methoxy-phenyl)-2-propenoic acid 2-methoxy-4-[(1E)-3-[4-(nitrooxy)butoxy]-3-oxo-1-propenyl]phenyl ester;
(nitrooxy-methyl)phenyl-2-hydroxybenzoate;
(E)-3-(3,4-Dihydroxy-phenyl)-2-propenoic acid 3-(nitrooxy methyl)phenyl ester;
(E)-3-(3,4-Dihydroxy-phenyl)-2-propenoic acid 2-(nitrooxy methyl)phenyl ester;
(E)-3-(3,4-Dihydroxy-phenyl)-2-propenoic acid 4-(nitrooxy methyl)phenyl ester;
(E)-3-(3,4-Dihydroxy-phenyl)-2-propenoic acid 3-(nitrooxy methyl)phenyl amide;
(E)-3-(3,4-Dihydroxy-phenyl)-2-propenoic acid 2-(nitrooxy methyl)phenyl amide;
(E)-3-(3,4-Dihydroxy-phenyl)-2-propenoic acid 4-(nitrooxy methyl)phenyl amide;
(E)-3-(3,5-Dihydroxy-phenyl)-2-propenoic acid 3-(nitrooxy-methyl)phenyl ester;
(E)-3-(3,5-Dihydroxy-phenyl)-2-propenoic acid 3-(nitrooxy-methyl)phenyl amide;
(E)-3-(3-hydroxy-4 methoxy-phenyl)-2-propenoic acid 3-(nitrooxymethyl)phenyl ester;
(E)-3-(3-hydroxy-4-methoxy-phenyl)-2-propenoic acid 3-(nitrooxymethyl)phenyl amide;
(E)-3-(3,4-Dimethoxy-phenyl)-2-propenoic acid 3-(nitrooxy-methyl)phenyl ester;
(E)-3-(3,4-Dimethoxy-phenyl)-2-propenoic acid 2-(nitrooxy-methyl)phenyl ester;
(E)-3-(3,4-Dimethoxy-phenyl)-2-propenoic acid 4-(nitrooxy-methyl)phenyl ester;
(E)-3-(3,4-Dimethoxy-phenyl)-2-propenoic acid 3-(nitrooxy-methyl)phenyl amide;
(E)-3-(3,4-Dimethoxy-phenyl)-2-propenoic acid 2-(nitrooxy-methyl)phenyl amide;
(E)-3-(3,4-Dimethoxy-phenyl)-2-propenoic acid 4-(nitrooxy-methyl)phenyl amide;
(E)-3-(4-hydroxy-3-methoxy-phenyl)prop-2-enoic acid;
(E)-3-(4-hydroxy-3-methoxy-phenyl)-2-propenoic acid 4-(nitrooxy)butyl ester;
(E)-3-(4-hydroxy-3-methoxy-phenyl)-2-propenoic acid 3-(nitrooxy-methyl)phenyl ester;
(E)-3-(4-hydroxy-3-methoxy-phenyl)-2-propenoic acid 6-(nitrooxy-methyl)-2-pyridinylmethylester hydrochloride;
(E)-3-(4-hydroxy-3-methoxy-phenyl)-2-propenoic acid 2-methoxy-4-[(1E)-3-[4-(nitrooxy)butoxy]-3-oxo-1-propenyl]phenyl ester;
3-(nitrooxy-methyl)phenyl-2-hydroxybenzoate.

10. Compounds according to claim 9 for use as medicaments.

11. Pharmaceutical composition comprising a compound according to claim 9 and a pharmaceutical acceptable excipient.
